(19) United States
(12) Patent Application Publication
(10) Pub. No.: US 2018/0220956 A1
(43) Pub. Date: Aug. 9, 2018

(54) Bruxism tracking and reduction device and methods

(71) Applicant: Peter Kuhar, Palo Alto, CA (US)

(72) Inventors: Peter Kuhar, Palo Alto, CA (US); Helena Jagodnik Kuhar, Palo Alto, CA (US)

(73) Assignee: Peter Kuhar, Palo Alto, CA (US)

(21) Appl. No.: 15/945,763

(22) Filed: Apr. 5, 2018

(51) Int. Cl. A61B 5/00

(52) U.S. Cl. CPC A61B 5/4557; A61B 5/6814; A61B 5/11; A61B 5/4547; A61B 5/486

(57) ABSTRACT

A device and method for tracking and reducing bruxism and related conditions. In one embodiment using an optical muscle activity sensor and a prediction based biofeedback method for reducing bruxism. The device can also be used to track bruxism by the user or a health professional. One embodiment is worn as a headband and positioned over the temporalis muscle with biofeedback provided directly to the temporalis muscle.

### CROSS-REFERENCE TO RELATED APPLICATIONS

This application claims the benefit of U.S. Provisional Application No. 62/48,250, filed on Apr. 6, 2018, the entire disclosure of which is incorporated by reference herein.

### TECHNICAL FIELD

The present invention related to biofeedback devices and muscle sensors, and more particularly an apparatus and method of tracking and reducing bruxism and related conditions.

### BACKGROUND OF THE INVENTION

### Field of the Invention

Teeth Grinding (bruxism) and clenching are a serious problem for a large percentage of the population. Causing teeth wear, cracks in teeth, tooth mobility, and headaches. The most common solution is to protect the teeth with a dental mouth guard. Which only protects from tooth damage, but does not alleviate headaches or prevent tooth mobility.

Some believe that lifestyle interventions can help with the problem. Things like alcohol and tobacco consumption, stress and anxiety are believed to be correlated with the problem. Without the means of a tracking the problem it's hard to know what intervention works and which does not.

Biofeedback is a promising method of bruxism reduction if properly implemented.

### Description of Prior Art

The most common way to monitor mastication muscle activity is using Electromyography (EMG). For example in device presented by Lee Weinstein et al in U.S. Pat. No. 5,270,466B1. The problem with using EMG to measure muscle activity is that it requires an electrical contact with the skin on top of the muscle. Electrical contact with the skin frequently caused adverse reactions like rashes and burns, this is particularly problematic since the device is worn for prolonged periods of time. Every night during sleep which on average 8 hours continuously.

EMG has other problems like the fact that EMG electrode contacts deteriorate over time and some devices even use single-use electrodes which is expensive if you use the device every night.

EMG electrodes also have a minimum size to provide good electrical contact with the skin, which makes miniaturization harder and thus makes the EMG based devices more uncomfortable to wear.

Different methods of biofeedback have been proposed in the past. Like electrical stimulation in patent US20120271190A1 which require electrical contact with the skin, this has similar problems as EMG. U.S. Pat. No. 6,270,466B1 proposes the use of an acoustic transducer which can disturb a partner sleeping in the same bed.

The other problem with previously mentioned biofeedback system is that they wait for the muscle activity to happen before applying stimulation. Which is too late since the force on the teeth was already exerted.

### SUMMARY OF THE EMBODIMENTS

One embodiment of the bruxism tracking and reduction device includes an optical muscle sensor first presented in this patent application and a visual and a vibratory stimulation device to provide bruxism reduction through biofeedback. A novel bruxism event prediction is used to predict and inhibit the bruxism event before it happens.

The device can be used for to reduce and treat bruxism as well as for tracking bruxism by the user or a health professional. The device is small and comfortable to wear.

### BRIEF DESCRIPTION OF THE DRAWINGS

FIG. 1 shows the user wearing the first embodiment.

FIG. 2 shows the first embodiment from the bottom size that touched the skin while wearing.

FIG. 3 shows positions of the light sources and photodetectors over a muscle.

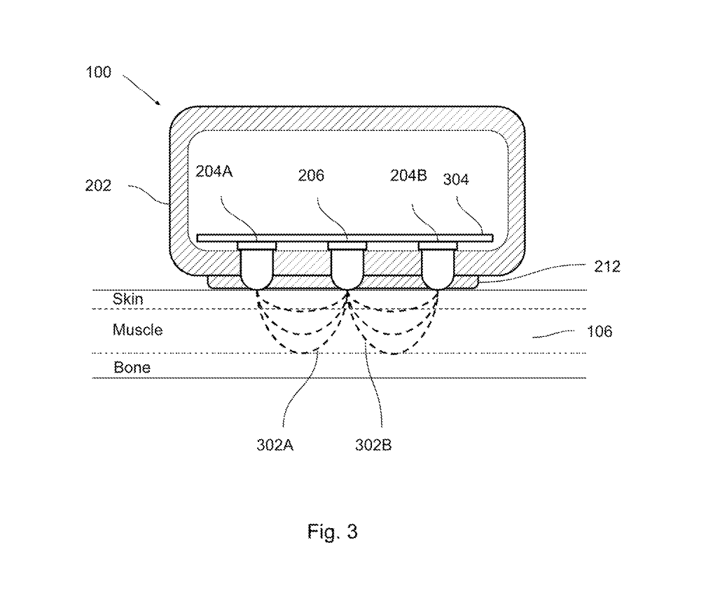

FIG. 4 shows a block diagram of the PCB assembly

FIG. 5 shows the algorithm flow diagram.

FIG. 6 shows characteristic waveforms during a bruxism event

### DRAWINGS—LIST OF REFERENCE NUMERALS

100 Device Assembly—bruxism tracking and reduction device
102 Elastic band
104 User
106 Temporalis muscle
202 Device enclosure
204A/B Dual wavelength infrared light source
206 Photosensor
208 Visual stimulation light source
210 Vibration motor
212 Sensor cluster
214 mounting slots
302A/B Transflectance through the muscle
304 PCB Assembly
402 Processor
404 LED Driver
406 Transimpedance amplifier
408 Motion sensor
410 USB connection
412 Power source
414 Wireless module
502A/B Sampled transflectance signal
504 heart pulse peak detector
506 heartbeat based normalization and DC removal
507A/B Aligned transflectance signals
508 Subtraction
509 Muscle activity
510 Bruxism pattern detector
511 Significant motion detector
512 Bruxism event predictor
513 Sleep detector
514 Bruxism biofeedback generator
602 Heart pulse

### DETAILED DESCRIPTION OF SPECIFIC EMBODIMENTS

### Detailed Description—First Embodiment

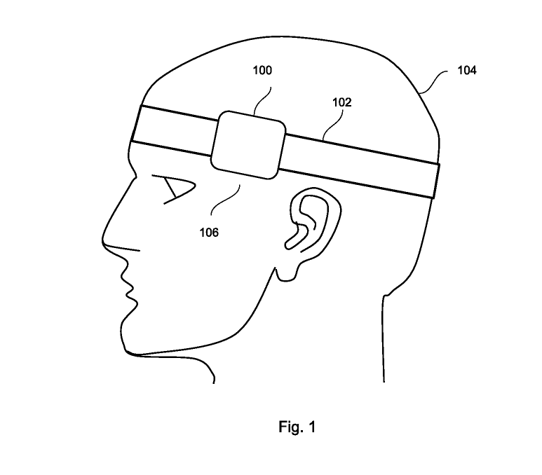

FIG. 1 shows the user wearing one embodiment of the bruxism tracking and reduction device assembly 100. In this embodiment, the device assembly 100 is affixed to the user 104 using an elastic band 102 in a way that the devices sensors cluster 212 is positioned over the temporalis muscle 106. The sensor cluster 212 is tightly coupled to the skin in a way that it prevents accidental movement while wearing.

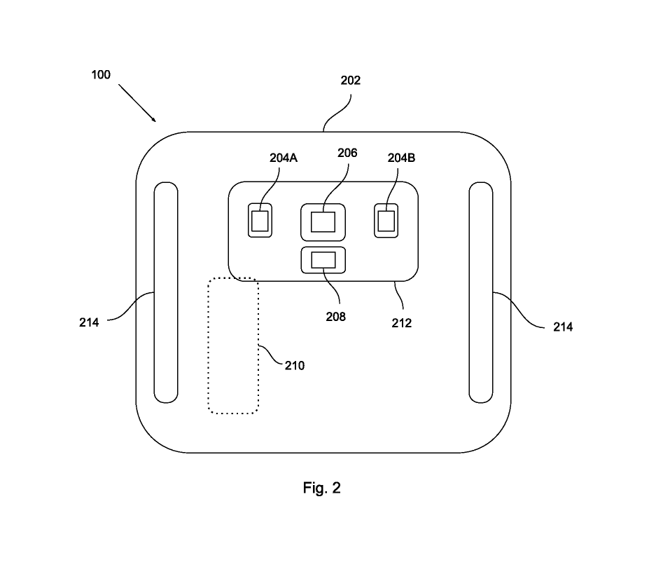

FIG. 2 shows the device assembly 100 from the bottom side which is touching the skin. The enclosure 202 is made out of opaque material that does not pass through light. The sensor cluster 212 is slightly raised over the overall enclosure 202 to provide better contact with the skin and prevent outside light from reaching the sensors. There is a photosensor 206 in the center of the sensor cluster 212, the photosensor 206 can be a photodiode, a CMOS camera or other means for measuring light intensity. On the sides of the photosensor 206, there are dual wavelength infrared light sources 204A/B that emit light at wavelengths 850 nm and 940 nm or other wavelengths that provide a different path through the muscle tissue depending on the wavelength. The light sources 204 are spaced approximately 1 cm from the photosensor 206. This provides good transflectance 302A/B through the temporalis muscle 106.

Visual stimulation light source 208 is an RGB LED diode in one embodiment. It's positioned in the sensor cluster 212 to provide good contact with the skin and prevent the light escaping. The stimulation light source 208 stimulates the optic nerve of the user 104 through the skull. Other embodiments are possible where the stimulation light source is position in front of the user's eyes or light source that is external to the device can be used.

The device assembly 100 is attached to the elastic band 102 through mounting slits 214.

Haptic or vibrational stimulation is provided through a vibration motor 210 which could be an eccentric mass vibration motor, a linear actuator, a piezo or other means of generation vibration.

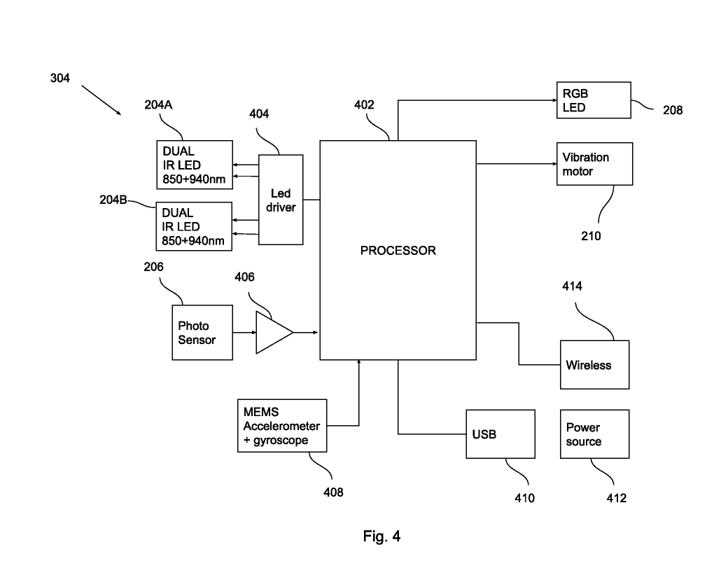

FIG. 4 shows the overview of the components on the PCB assembly 304. The main part is a processor 402 which in one embodiment is a low power microcontroller which includes memory, ADC and DAC converters etc. For example, STM32L443 manufactured by ST Microelectronics. The led driver 404 is connected to the light sources 204A/B and allows the processor 402 to control the current through the light sources 204 individually. The photosensor 206 is connected the processor 402 through a transimpedance amplifier 406 which converts electrical current to voltage. Other embodiments might a monolith solution which includes all sensor components in one part.

A motion sensor 408, which could be a MEMS sensor unit that includes an accelerometer and a gyroscope is also connected the processor 402.

The PCB assembly also has a power source 412, a USB connection 410 an a wireless module 414.

### Operation—First Embodiment

The first embodiment measures a plurality of biological signals like muscle activity, heart pulse, and motion and provides bruxism activity information to the users and reduces bruxism using a biofeedback method.

The novel optical muscle sensor works by measuring thickness changes of the temporalis muscle 106 caused by muscle contractions. It measures transflectance 302A/B through the muscle at two different wavelengths and compares them. The transflectance through skin and muscle is affected by blood pressure changes caused by heart pulse and muscle contractions. The ratio of the contribution to the transflectance change depends on the wavelength. Thus if we measure it at different wavelengths we can algorithmically determine the contribution of each.

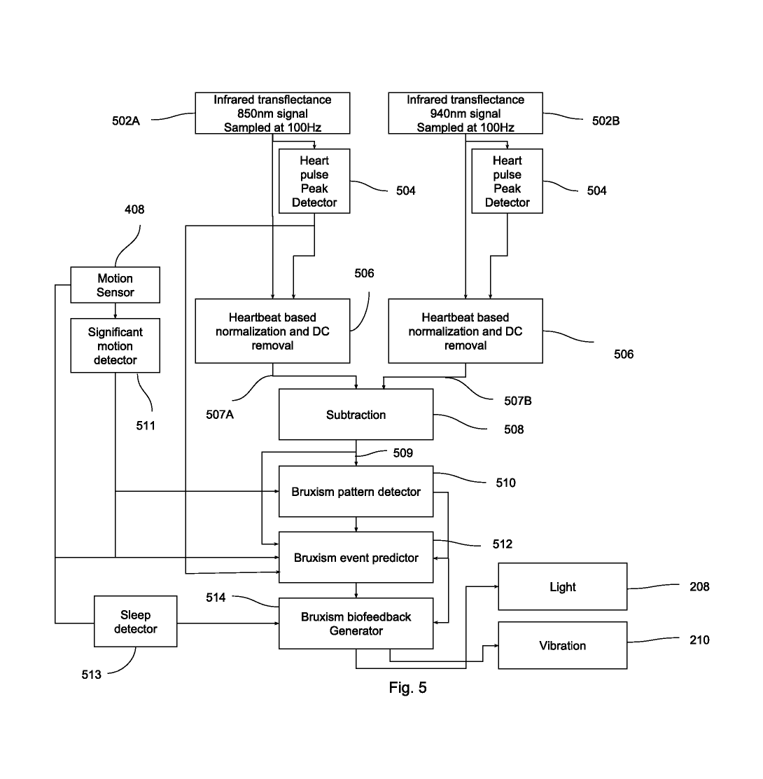

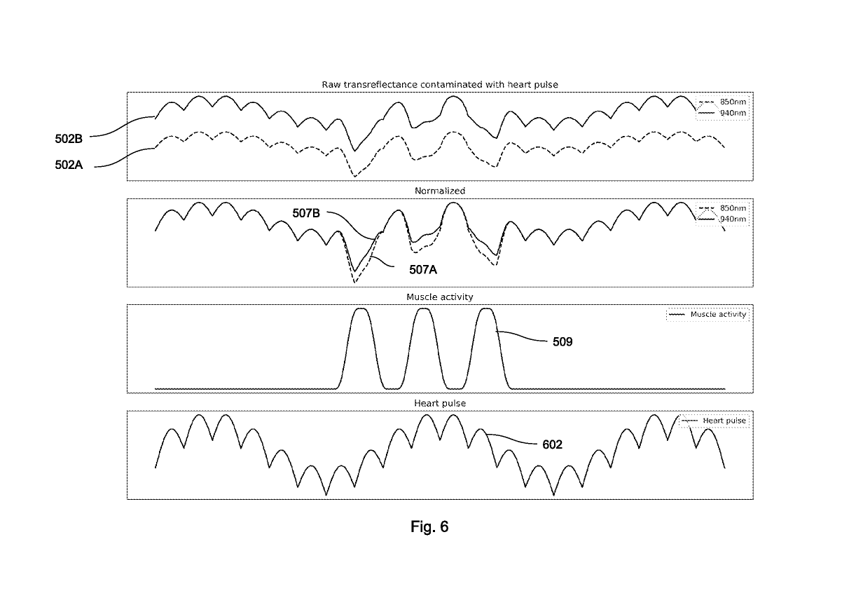

The transflectance 302A/B is sampled at regular interval for example at 100 Hz providing the signal samples 502A/B on FIG. 5 and FIG. 6. FIG. 6 show the typical signal waveforms during a bruxism event. The signals 502 have different gains and DC offsets and need to be aligned before they can be used. The aligned transflectance signals 507A/B can be then be subtracted to get the muscle activity signal 509. Other embodiments could use Independent Component Analysis or similar methods to extract the muscle activity 509 and the heart pulse signal 602.

One way to extract the muscle activity 509 and detect and predict bruxism events is presented in FIG. 5. The transflectance signals 502A/B are first passed through a heart pulse peak detector 504 which extract peaks and valleys of the pulse using methods also used in pulse oximetry devices. This provides the gains for heartbeat based normalization and DC removal 506 which is effectively aligning the signals 502A/B into 507A/B. The difference between the signal is then calculated using subtraction 508 which gives the muscle activity signal 509.

This gives the base for the novel optical muscle sensor. Using light sources 204A/B and photosensor 206 and producing a muscle activity signal 509 using the method presented in part of the FIG. 5.

The samples from the motion sensor 408 are first passed to significant motion detector 511 which senses user's 104 movement that might contaminate the transflectance 302A/B signals.

The muscle activity 509 is then passed to a bruxism pattern detector 510 together with the motion data from significant motion detector 511. The bruxism pattern detector 510 in one of the embodiments is a recurrent neural network trained on labeled bruxism event data from this or a similar device.

The other novel innovation is a bruxism event predictor 512 which predicts the bruxism event before it happens, based on the data from all the available sensors. The motion sensor 408, the heart pulse from the heart pulse peak detector 504 and previous data from the bruxisms pattern detector 510. The bruxism event predictor 512 is a recurrent neural network trained on labeled bruxism events collected for this or similar device.

The biofeedback method of bruxism reduction uses visual and/or vibrational means of stimulation. The bruxism biofeedback generator 514 generates a stimulation when a start of a bruxism event is detected by the detector 510 or when a bruxism event is predicted by the bruxism event predictor 512. The signal from the actigraphy-based sleep detector 513 is used to inhibit the stimulation when the user 104 is not asleep. The bruxism biofeedback generator 514 adjusts and personalizes biofeedback parameters based on the effectiveness of preceding biofeedback stimulations. A plurality of biofeedback strategies can be set over the wireless or USB connection.

A combination of a vibration motor 210 and a visual stimulation light source 208 is used so that the bruxism biofeedback generator can assess the effectiveness of biofeedback-based on the result from the bruxism pattern detector 510 and personalizes the biofeedback to the user. The vibration generated by the vibration motor 210 is chosen from the range of approximately 15 Hz and 250 Hz and positioned in a way that it stimulates the temporalis muscle 106 and the tendons connecting the temporalis muscle to the jaw. This vibration signals the brain to open the jaw subconsciously. The duration of the stimulation is typically 0.5 to 2 seconds.

Another method of stimulation like sound, heat, direct transcranial current stimulation or other brain stimulation methods could also be used.

The device 100 has a USB connection 410 and/or a wireless module 414 to transfer the bruxism event and biofeedback data to an external system like a mobile phone where the effectiveness can be evaluated by the user 104 or others like a trained sleep or dental professional.

Thus this embodiment presents several novel and important advantages over the existing methods and devices.

The optical-based muscle sensor is more reliable and comfortable to use compared to EMG based sensor since it does not require direct contact with the skin.

The biofeedback method uses the bruxism event prediction which can predict a bruxism event before it starts and inhibits it before it happens.

The visual stimulation light source 208 is positioned in a way that it stimulates the optic nerve through the skull. Thus the light does not disturb a partner sleeping in the same bed.

The vibration motor 210 stimulates the temporalis muscle 106 and it's tendons directly to signal the subconscious brain to open the jaw without waking up the user. While the previous methods used vibration as a means to alert or wake up the user when bruxism was detected.

Although the description above contains many specifics these should not be construed as limiting the scope the embodiments but merely providing an illustration of some possible embodiments.

For example, in another embodiment, the device may be positioned over a different mastication muscle like the masseter muscle. Instead of the elastic band, the device can be secured to the user using different means, like using an adhesive. Instead of an optical muscle sensor, an acoustic or vibratory muscle sensor could be used to measure muscle contraction. The optical muscle sensor could use a different method to extract the muscle signal, for example, principal component analysis, independent component analysis, or a least squares minimizer. The bruxism event detector and predictor can be implemented in ways other than using a recurrent neural network, it could use other types of neural networks or be handcrafted code based on the feature of the bruxism event.

The presented embodiment has two multi-wavelength light sources for redundancy. But other combinations of light sources and photosensors are possible providing one or more transflectance measurements across two or more wavelengths.

The bruxism event predictor 512 could also use a plurality of other biological signals like breathing rate, brain activity from a near-infrared spectroscopy based brain sensor or an EEG sensor, blood oxygenation etc.

What is claimed is:

1. A method for tracking and reducing bruxism, the method comprising:
measuring a mastication muscle activity;
measuring a plurality of biological signals;
generating a prediction of a bruxism event based on preceding data samples of said biological signals, and preceding data samples of said mastication muscle activity;
generating a stimulation based on said bruxism event prediction, said mastication muscle activity;
whereby said bruxism is reduced.

2. A method for tracking and reducing bruxism from claim 1, wherein said stimulation is visual.

3. A method for tracking and reducing bruxism from claim 1, wherein said stimulation is a vibration.

4. A method for tracking and reducing bruxism from claim 1, wherein said prediction of the bruxism event is generated by a neural network.

5. A method for tracking and reducing bruxism from claim 1, further comprising:
adjusting said stimulation based on the effectiveness of preceding stimulations.

6. A method for tracking and reducing bruxism from claim 1, further comprising:
adjusting said stimulation based on the effectiveness of preceding stimulations of other users.

7. A Bruxism reduction device, comprising:
a mastication muscle activity sensor;
a processor;
a means for measuring a plurality of biological signals;
a means for stimulation;
wherein said processor implements a bruxism event prediction algorithm that uses said biological signals, and said mastication muscle activity to generate a bruxism event prediction, before said bruxism event happens;
said processor implements a bruxism reduction algorithm that uses said bruxism event prediction, said mastication muscle activity to stimulate the user using said means for stimulation;
whereby bruxism is reduced.

8. A Bruxism reduction device from claim 7 wherein said bruxism event prediction algorithm is a neural network.

9. A Bruxism reduction device from claim 7 wherein said bruxism reduction algorithm has a means for adjusting parameters of said means for stimulation based on the effectiveness of prior stimulations.

10. A Bruxism reduction device from claim 7 wherein said means for stimulation is a vibration generation device.

11. A Bruxism reduction device from claim 7 wherein said means for stimulation is a means for generating sound.

12. A Bruxism reduction device from claim 7 wherein said means for stimulation is a light source.

13. A Bruxism reduction device from claim 7 wherein said means for stimulation is a light source device positioned on a user in a way that said light stimulates an optic nerve through users skull.

14. A Bruxism reduction device from claim 7 wherein said bruxism device is securely attached to a user using an elastic band.

15. A muscle activity sensor, comprising:
a one or more light sources;
a one or more photosensors;
a processor;
wherein said processor is connected to said light sources and said photodetectors and said light sources and said photodetectors are positioned in a way that a transflectance through said muscle can be measured at two or more different wavelengths;
said processor uses a means for calculating muscle position changes from changes in said transflectance at different wavelengths;
whereby said muscle activity is measured.

16. A muscle activity sensor from claim 15 wherein said light sources emit infrared light.

17. A muscle activity sensor from claim 15 wherein said light sources emit infrared light at wavelengths 850 nm and 940 nm.

18. A muscle activity sensor from claim 15 wherein said means to calculate muscle position changes is a neural network.

19. A muscle activity sensor from claim 15 wherein said means for calculating muscle position changes is an algorithm that uses said transflectance preceding said muscle activity to align and subtract said transflectances.

20. A muscle activity sensor from claim 15 wherein said means for calculating muscle position changes is an algorithm that uses Independent Component Analysis.
